(19) United States
(12) Patent Application Publication
(10) Pub. No.: US 2022/0401665 A1
(43) Pub. Date: Dec. 22, 2022

(54) PORTABLE LOOSE-LEAF MATERIAL VAPORIZER

(71) Applicants: Organicix, LLC, Las Vegas, NV (US); Cansativa Medical Devices GmbH, Neuenahr-Ahrweiler (DE)

(72) Inventors: Cortney Smith, Las Vegas, NV (US); Josh WANG, Las Vegas, NV (US); Markus HALLER, Bad Neuenahr-Ahrweiler (DE); Alexander WEGENER, Bad Neuenahr-Ahrweiler (DE); Christan HAUPTMANN, Bad Neuenahr-Ahrweiler (DE)

(73) Assignees: Organicix, LLC, Las Vegas, NV (US); Cansativa Medical Devices GmbH, Neuenahr-Ahrweiler (DE)

(21) Appl. No.: 17/755,717

(22) PCT Filed: Dec. 8, 2020
(86) PCT No.: PCT/EP2020/085091

§ 371 Date: May 5, 2022

Related U.S. Application Data
(60) Provisional application No. 62/945,597, filed on Dec. 9, 2019.

(51) Int. Cl. A61M 11/04; A24F 40/20; A24F 40/57; A24F 40/51; A24F 40/48; A24F 40/60

(52) U.S. Cl. CPC A61M 11/042; A24F 40/20; A24F 40/57; A24F 40/51; A24F 40/48; A24F 40/60; A61M 2205/3368; A61M 2205/3334

(57) ABSTRACT

A portable loose-leaf material vaporizer 1 comprises a heating chamber 20 for housing loose leaf material and vaporizing one or more active agents from the loose-leaf material, a mouthpiece 3 for withdrawal of the one or more active agents vaporized from the loose-leaf material, and a processing unit. The processing unit is configured to control temperature of the heating chamber 20 and to estimate a dosage of the one or more active agents withdrawn from the vaporizer 1 based on a mathematical model. The mathematical model relates vapor generating time and loose-leaf material properties, and optionally temperature of the heating chamber 20, to the dosage of the one or more active agents withdrawn or to be withdrawn from the vaporizer 1.

The present invention lies in the field of vaporizers. More particular, the invention relates to a portable loose-leaf material vaporizer comprising a heating chamber for housing loose-leaf material and vaporizing one or more active agents from the loose-leaf material, a mouthpiece for withdrawal of the one or more active agents vaporized from the loose-leaf material, and a processing unit, wherein the processing unit is configured to control temperature of the heating chamber.

Vaporizers of this kind are known in the art. An example is the IQ from DaVinci. The vaporizer is for primary use with loose leaf cannabis and secondary use with concentrates of cannabis. Cannabis is a flowering plant most often consumed in its ‘loose-leaf’ or flower form or in a variety of concentrated forms and purchased legally in many countries at dispensaries. Two active agents are of medical interest: delta-9-tetrahydrocannabinol (“THC”) and cannabidiol (“CBD”), which belong to the class of cannabinoids. THC is the psychoactive component within the plant, causing the ‘high’ commonly associated with its use. CBD is a form of THC but acts as a pain relieving rather than a psychoactive agent. Both THC and CBD appear in precursor forms tetrahydrocannabinolic acid (“THCA”) and cannabidiolic acid (“CBDA”), respectively, and are converted to their active forms upon heating known as decarboxylation or activation. The vaporizer heats the loose-leaf cannabis generating vapor that contains THC and/or CBD. Users inhale through the device to simultaneously withdraw and consume the vapor.

The IQ is provided with a smartphone app that allows to set the temperature and/or customize the temperature or temperature profile to allow for adapting the vaping experience. However, for use of the vaporizer in medicine, it would be desirable to control the dosage withdrawn from the vaporizer. In particular, it would be desirable to be able to control dosage without having to employ pre-measured containers of cannabis oil, limiting their heating mechanisms by an approximation of mass loss and thus cannabinoid consumption. Moreover, it would be desirable to provide a vaporizer that does not necessitate an application run on a terminal device for controlling the dosage.

This problem is solved by a vaporizer of the aforementioned type implementing dosage estimation functionality as disclosed herein.

Further objectives become apparent from the following description and especially from the described advantages.

The Dosage Estimation Functionality

According to a first aspect of the present invention, a vaporizer of the aforementioned type comprises a processing unit configured to estimate a dosage of the one or more active agents withdrawn from the vaporizer based on a mathematical model, wherein the mathematical model relates vapor generating time and loose-leaf material properties, and optionally temperature of the heating chamber, to the dosage of the one or more active agents withdrawn or to be withdrawn from the vaporizer.

The first aspect of the present invention is based on the innovation of the inventors that the dosage that is withdrawn from the vaporizer is largely dependent on the vapor generating time, the loose-leaf material properties, in particular the amount of the one or more active agents contained in the loose-leaf material within the heating chamber, and the temperature of the heating chamber, whereas other influences, including user-specific or strain-specific influences other than the amount of the one or more active agents, can be ignored to decrease complexity of the model and yet obtain reasonable estimations of the withdrawn dosage. One key assumption includes that all active agent(s) vaporized within the device during the vapor generating time is (are) withdrawn from the device. Thereby, the dosage withdrawn from the vaporizer can be estimated. The dosage estimation provides the basis for dosage control. Accordingly, it is preferred that the processing unit is configured to further control the dosage of the one or more active agents withdrawn from the vaporizer.

The mathematical model is preferably a calibrated mathematical model. A calibrated mathematical model is a model implementing empirical data from reference samples so that the model yields an output based on known input(s). For instance, the empirical data includes data on the production rate of the one or more active agents in dependency of the loose-leaf properties. An accordingly calibrated mathematical model would then include a relationship between the loose-leaf properties, the dosage and the vapor generating time. As an advantage, the model is very simple and adequate for a vaporizer that is used at the same or close to the temperature at which the calibration experiments have been conducted (reference temperature). It is further preferred that the mathematical model is a validated mathematical model. i.e. a model validated using experimental data.

If the vaporizer was intended to be used distant from said reference temperature or at different temperatures (e.g. because it allows for setting different temperatures and/or temperature profiles or the heating chamber), the estimation error would increase with an increasing deviation from the reference temperature. To overcome this disadvantage, it is preferred that the empirical data include data on the production rate of the one or more active agents in dependency of the loose-leaf properties and the temperature of the heating chamber. The calibrated mathematical model can then, for instance, be used to estimate how much active agent(s) of a given loose-leaf material is (are) produced in a certain period of time, at a certain temperature. Moreover, the mathematical model would be able to compute appropriate combinations of the temperature of the heating chamber and the vapor generating time so that a predetermined dosage becomes available in the vapor for withdrawal.

The term “loose-leaf material” is synonymous to a dry herb material and describes a plant material provided in particulate form comprising leafs and/or flowers. The loose-leaf material comprises one or more active agents. Preferably, the loose-leaf material is cannabis and the one or more active agents include THC and/or CBD. When reference is made in the following to cannabis and THC/CBD, corresponding embodiments are meant to be described for a loose-leaf material and one or more active agents generally.

The “loose-leaf material properties”, as understood herein, are information that define the amount of the one or more active agents contained in the loose-leaf material. Suitable information in this regard are the content of the one or more active agent(s) relative to the loose-leaf material and the amount of loose-leaf material contained in the heating chamber. Both information can be easily determined. For instance, the relative content of the one or more active agents, in particular the THC and/or CBD content, is regularly printed on marketed products.

The “vapor generating time” denotes the time over which the loose-leaf material is heated in the heating chamber at or above the boiling point of the one or more active agent(s). The boiling point of THC is 157° C. The boiling point of CBD is 180° C. Because of the small quantities of THC and CBD that evaporate, it can be assumed, that the already decarboxylated amounts of THC and CBD immediately evaporate and that any newly produced THC and CBD also immediately are available in the gaseous (vapor) phase. Evaporation stops immediately, once the temperature is reduced to values below the boiling points. Since the decarboxylation rate is temperature-dependent, the mathematical model may implement the fact that the rate with which gaseous THC and CBD is formed increase with increasing temperature of the heating chamber.

If the loose-leaf material contains more than one active agent, and the dosage for more than one active agent is of interest, the mathematical model may comprise equations for each active agent of interest.

With the aforementioned values, a mathematical model can be created to relate loose-leaf material properties, the vapor generating time, optionally the temperature of the heating chamber, and the dosage. The term “relating”, as used herein, refers to a mathematical relationship between one or more input variable(s) and one or more output value(s). A suitable mathematical relationship includes a case, where the dosage increases with an increasing amount of the one or more active agent(s) contained in the loose-leaf material and where the dosage increases with an increasing vapor generating time and, optionally, where the dosage increases with an increasing temperature of the heating chamber.

The input variables may include the loose-leaf material properties. The output values may define how much active agent(s) of the given loose-leaf material is (are) produced in a certain period of time, at a certain temperature. Alternatively, the input variables may include the loose-leaf material properties and the dosage to be withdrawn. In this case, the output values may include one or more appropriate combinations of the vapor generating time and the temperature so that the dosage intended to be withdrawn is produced in the one or more respective combinations of the vapor generating time and the temperature.

In a preferred embodiment, the estimation is performed continuously. The estimation may for instance be performed every 0.1 second, or every second. Accordingly, the term “dosage”, as understood herein, preferably refers to the real-time delivery of THC and/or CBD consumption information to a user. The estimation may also be performed repeatedly, for instance after each draw.

In a further preferred embodiment of the present invention, the processing unit is configured to determine, based on the mathematical model, one or more of the following:
(i) the vapor generating time (i.e. time for heating the heating chamber to or above the boiling point(s) of the one or more active agent(s)) until a predetermined dosage is available for withdrawal from the vaporizer and/or
(ii) the temperature or temperature profile of the heating chamber so that a predetermined dosage is available within a predetermined vapor generating time for withdrawal from the vaporizer and/or
(iii) the number of draws until a predetermined dosage is withdrawn from the vaporizer.

In a further preferred embodiment of the present invention, the processing unit is configured to stop heating of the heating chamber and/or start cooling of the heating chamber when the processing unit determines that the predetermined dosage is available for withdrawal from the vaporizer. For example, once the predetermined dosage is available, i.e. determined to be present in the vapor phase, the heating is stopped so that a user can withdraw the one or more active agent(s) in the predetermined dosage. The overall dosage may also be split among a plurality of, e.g. 3, 4, 5 or 6, dosages. Once the first dosage is available, i.e. determined to be present in the vapor phase, the heating is stopped until a user has taken the draw. Afterwards, the heating is started and continued until the second dosage is available, etc.

In a further preferred embodiment of the present invention, the processing unit is configured to indicate to a user when a predetermined dosage is available for withdrawal from the vaporizer. In other terms, the vaporizer indicates its readiness for the next draw. For instance, the processing unit may actuate an LED lamp and/or a vibration mechanism. Thereby, it can be prevented that a user takes a draw too early, i.e. at a time when the predetermined dosage has not yet been activated (e.g. THCA and/or CBDA decarboxylated) and/or transferred into the vapor phase. In this way, it can be safeguarded that a user indeed withdraws the predetermined dosage.

### The Flow Detector

According to the second aspect of the present invention, a flow detector is implemented in order to detect the flow through the vaporizer.

Accordingly, the second aspect of the present invention pertains to a portable loose-leaf material vaporizer, preferably a portable loose-leaf material vaporizer according to the first aspect, comprising:
a heating chamber for housing loose leaf material and vaporizing one or more active agents from the loose-leaf material,
a mouthpiece for withdrawal of the one or more active agents vaporized from the loose-leaf material,
a processing unit, and
a flow detector for detecting flow through the vaporizer
wherein the processing unit is configured to control temperature of the heating chamber and
wherein the processing unit is preferably configured to estimate a dosage of the one or more active agents withdrawn from the vaporizer based on a mathematical model,
wherein the mathematical model preferably relates vapor generating time and loose-leaf material properties, and optionally temperature of the heating chamber, to the dosage of the one or more active agents withdrawn or to be withdrawn from the vaporizer.

The term “flow detector” as used herein refers to a detector that is capable to detect whether or not flow occurs through the vaporizer. By means of the flow detector, the processing unit can determine when vapor containing the one or more active agent(s), preferably a predetermined dosage thereof, made available in the vapor has been withdrawn from the vaporizer. Thereby it is possible to determine when the vaporizer can be prepared for a next draw. This is particularly useful when an overal dosage is split among a plurality of dosages to be withdrawn in individual draws.

Moreover, the flow detector enables to determine the inhalation duration from the start and end of the inhalation. Although it can well be assumed that all active agent(s) vaporized within the device during the vapor generating time is (are) withdrawn from the device, the reliability and accuracy of the dosage estimation may be further enhanced by taking into account the inhalation duration. Model accuracy could be expected to be improved especially for users taking relatively large or fairly small draws as compared to an average user. Accordingly, a vaporizer of the second aspect of the invention is preferred, wherein the mathematical model (further) relates inhalation duration to the dosage.

In a preferred embodiment of the second aspect of the invention the flow detector is arranged outside a flow path directly connecting the heating chamber and the mouthpiece. Preferably, the flow detector is arranged in a dead-end branch branching from the flow path connecting the chamber and the mouthpiece. The advantage of placing the flow detector outside a flow path directly connecting the heating chamber and the mouthpiece is that fouling can be avoided. In turn, when the flow detector is placed within the flow path directly connecting the heating chamber and the mouthpiece, oily and sticky components evaporated from the loose-leaf material tend to deposit on the flow detector, thereby deteriorating the detector's reliability and/or life.

To keep the vaporizer small, the flow detector should be as small as possible. Thus, according to a further preferred embodiment, the flow detector is selected from the group consisting of differential pressure sensors, capacitive air flow sensors, spinning fans/turbines, moving flap-type sensors, temperature sensors and thermal flow sensors. Preferably, the flow detector is a diaphragm pressure sensor. Other flow detectors exist but are too large. The detectors disclosed herein can advantageously be integrated into the portable vaporizer.

The concept by which the aforementioned flow detectors work is known to a person skilled in the art and briefly described in the following.

Differential pressure sensors: Measurement style: Direct/Indirect. Description: A small sensor that measures pressure at 2 locations—a significant difference in those measurements signifies flow. Output: Occurrence and/or intensity of flow.

Capacitive air flow sensors: Measurement style: Indirect. Description: A small diaphragm flexes when pressure drops on one side of it as a result of flow. The change in geometry causes a change in capacitance which signifies flow. Output: Occurrence.

Spinning fans/turbines: Measurement style: Direct. Description: A small fan is placed in the airpath. User inhalation spins the fan which signifies flow. Output: Occurrence and intensity.

Moving flap-type sensors: Measurement style: Direct. Description: Similar to a spinning fan/turbine. User inhalation pushes the flap which signifies flow. Output: Occurrence and intensity.

Temperature sensors: Measurement style: Direct/Indirect. Description: Only applicable when there is a change in temperature—a significant difference in temperature between the initial measurement and the final measurement signifies flow. Output: Occurrence and intensity.

Thermal flow sensors: Measurement style: Direct. Description: A small heater is positioned between an upstream and a downstream temperature sensor. User inhalation heats the downstream sensor. The difference between the upstream and downstream sensor signifies flow. Output: Occurrence and intensity.

The Flow Regulating Element (“Air Dial”)

A third aspect of the present invention pertains to a portable loose-leaf material vaporizer, preferably the portable loose-leaf material vaporizer according to the first or second aspect, the vaporizer comprising:
a heating chamber for housing loose leaf material and vaporizing one or more active agents from the loose-leaf material,
a mouthpiece for withdrawal of the one or more active agents vaporized from the loose-leaf material,
a processing unit, and
a flow regulating element configured to exert one among a plurality of resistances against flow through the vaporizer, and
optionally a flow detector for detecting flow through the vaporizer,
wherein the processing unit is configured to control temperature of the heating chamber and
wherein the processing unit is preferably configured to estimate a dosage of the one or more active agents withdrawn from the vaporizer based on a mathematical model,
wherein the mathematical model preferably relates vapor generating time and loose-leaf material properties, and optionally temperature of the heating chamber and/or the one resistance exerted by the flow regulating element against the flow through the vaporizer, to the dosage of the one or more active agents withdrawn or to be withdrawn from the vaporizer.

The term “flow regulating element” also referred to herein as “air dial”, denotes an element that is capable of exerting a resistance against the flow through the vaporizer. Furthermore, the resistance can be chosen among a plurality of different resistances. Preferably, the plurality of resistances is determined by a plurality of positions of the flow regulating element relative to a flow path through the vaporizer. The flow regulating element is preferably located downstream of the heating chamber and restricts the flow or ambient air entering the flow path through the vaporizer to different degrees. The possibility to choose one among a plurality of resistances has the advantage that the vaping experience can be adjusted to personal preferences. A high resistance is associated with big “clouds”, i.e. concentrated vapor that comprises relatively little ambient air, whereas a low resistance leads to vapor “diluted” with ambient air. Furthermore, according to subjective judgement, big clouds are sometimes considered to lead to a higher “high”. Such subjective judgement does however not necessarily reflect a dosage effect. Quite to the contrary, the inventors rather assume that all active agent(s) vaporized within the device during the vapor generating time is (are) withdrawn from the device. Therefore, the resistance is considered to have no or only a small effect on the amount of active agent(s) withdrawn from the vaporizer, which assumption is confirmed by the inventors' studies described further below. Therefore, the resistance could be implemented in the mathematical model but is preferably neglected for dosage estimation for simplicity reasons.

According to a preferred embodiment, the flow regulating element comprises a rotatable disk. The rotatable disk preferably comprises a first section, which when rotated into a flow path of the vaporizer defines a first effective cross sectional flow area. The rotatable disk preferably further comprises a second section, which when rotated into the flow path of the vaporizer defines a second effective cross sectional flow area different from the first cross sectional flow area. The rotatable disk may comprise a further section defining a further effective cross sectional flow area different from the first and second cross sectional flow areas, and so on. Preferably, the number of sections defining different effective cross sectional flow areas is 3, 4, 5, 6, 7, 8, 9, or 10.

According to a further preferred embodiment of the present invention, the processing unit is configured to control the resistance exerted by the flow regulating element against the flow through the vaporizer. For example, the flow regulating element can be moved by a motor such as a rotary actuator. Advantageously, the motor may include a sensor for position feedback. Preferred in this regard is a servomotor. An advantage of such control functionality is that the flow of ambient air entering the vaporizer can automatically be increased when the temperature of the inhaled vapor has been determined to be too hot, i.e. at or above a predetermined temperature (see above).

Further Sensors

The vaporizer of the present invention may comprise one or more (further) sensors, as described in the following.

One useful sensor is a sensor for determining the resistance exerted by the flow regulating element against the flow through the vaporizer. Especially when the resistance is reflected by a definite position of the flow regulating element relative to a flow path through the vaporizer, as described above, there are a number of methods available to determine this position in addition to a servomotor including a sensor for position feedback. The methods include without limitation contact pin methods, continuous connection methods, wireless methods, optical methods, and sound methods. The concept by which these methods work becomes apparent by the following details. It should however be noted that additional features that are not required for determining the resistance exerted by the flow regulating element are described to aid understanding, thus optional and could be omitted for the purposes of the present invention.

Contact pin method: contact pins are mounted on the device body and the bottom cap where the air dial is located. When the cap is closed, the contact pins touch and can transmit a signal between the device body and the bottom cap. Turning the air dial can change a resistive or capacitive element which is measured by the device body, signaling the position of the air dial.

Continuous connection method: an electrical connection is continuously maintained through a wire that connects the device body and the bottom cap where the air dial is located. Turning the air dial can change a resistive or capacitive element which is measured by the device body via the connection, signaling the position of the air dial.

Wireless method: a variable size antenna is mounted on the air dial assembly. Turning the air dial changes the properties of the antenna. A wireless sensor in the device body detects the antenna size, signaling the position of the air dial.

Optical method: an optical/proximity sensor is mounted within the device body and faces the bottom cap. Turning the air dial exposes different physical features to the device body. The sensor detects these changes, signaling the position of the air dial.

Sound method: the air dial is configured to emit different frequencies at different positions during an inhalation. A sound sensor mounted on the device body detects the different frequencies, signaling the position of the air dial.

Another useful sensor is a temperature sensor. Accordingly, preferred is a vaporizer as described herein comprising one or more, preferably one or two, temperature sensor(s).

The temperature sensor(s) are preferably located adjacent to the heating chamber and/or adjacent to the mouthpiece. A temperature sensor adjacent to the heating chamber enables to accurately determine the temperature which the loose leaf material in the heating chamber is exposed to. Thereby, it is possible to accurately determine the processes, in particular the rate, by which the active agent(s) are formed (e.g. THCA and/or CBDA decarboxylation) and/or transferred into the gas (vapor) phase. A temperature sensor adjacent to the mouthpiece provides a good estimate about the temperature of the inhaled vapor. Moreover, it contributes to preventing a user to inhale too hot vapor. For instance, when the processing unit determines that the temperature detected by the temperature sensor is at or above a predetermined threshold, the temperature of the heating chamber is lowered, e.g. by allowing more ambient air to enter the vaporizer, or the heating chamber is switched off. Particular useful is the temperature sensor(s) when the vaporizer contains the flow regulating element. This is because the more ambient air enters the vaporizer, the more the temperature of the vapor drops, and vice versa.

Input Variables

The vaporizer of the present may implement dosage estimation based on one or more input variables. The input variable(s) may be either input by a user or determined by the processing unit via one or more sensors/detectors as described herein.

In accordance with this, it is preferred that the vaporizer comprises an interface for receiving sensor data (including data from detectors as described herein) and/or user input data. The user input data may be received from an application (e.g. app, PC program, web app) run on a terminal device such as a smartphone, tablet PC or PC, preferably via a wireless connection. The interface for receiving user input data may also be a user interface in which a user can directly input user input data. The user interface may comprise a plurality of actuation means, in particular buttons. While communication with a terminal device provides a more convenient solution, a user interface does not require an application or an additional device in order to input data and exploit the concept according to the present invention.

In a further preferred embodiment of the present invention, the sensor data and/or user input data include one or more of the following:
(i) a predetermined dosage or the one or more active agents to be withdrawn from the vaporizer (i.e. the dosage a user intends to take); and/or
(ii) a number of draws to be taken from the vaporizer (i.e. the number of draws among which the dosage is split); and/or
(iii) loose-leaf material properties (e.g. cannabis variety and/or percentage of active agent(s), weight of cannabis loaded into the heating chamber, status of the loose-leaf material, e.g. whether it has already been heated); and/or
(iv) information on a resistance exerted by a flow regulating element against the flow through the vaporizer (e.g. position of the “air dial”); and/or
(v) a temperature of the heating chamber.

Preferably, (i) and/or (ii) and/or (iii) is input by the user, i.e. received from the interface for receiving user input data. (iv) may be input by the user or determined by the processing unit based on the resistance exerted by the flow regulating element against the flow through the vaporizer, preferably based on a position of the flow regulating element, i.e. by the sensor for determining the resistance exerted by the flow regulating element against the flow through the vaporizer. (v) may be input by the user or determined by the processing unit based on the temperature of the heating chamber, i.e. by the one or more temperature sensor(s).

In order to optimize the dosage estimation, the control of the temperate of the heating chamber by the processing may be adapted to user-specific parameters. The user-specific parameters may include, for instance, a user's reaction time (i.e. time from draw indication to actual draw). The reaction time may serve to better control the moment, when the heating chamber needs to be turned off. Another example is a user's draw intensity, which may serve to decide on the best timing to turn off the heating chamber. The user-specific parameters can be detected as sensor data by the sensor(s), e.g. the flow detector, as disclosed herein, and stored and/or processed by the processing unit. The user-specific parameters may be defined for a user once, but may also be refined in an iterative manner. Consideration of user-specific parameters is of great advantage in order to cope with interpatient variability.

The sensor data (including user-specific parameters) and/or user input data may be stored in a storage unit comprised in the vaporizer or in a terminal device.

The functionalities of the vaporizers of the present invention as disclosed herein can be translated into corresponding uses and methods, which are encompassed by the present invention.

These and other aspects and embodiments of the invention will become apparent from and elucidated with reference to the embodiments described hereinafter taken in conjunction with the accompanying figures. Further advantages will be apparent to those of ordinary skill in the art upon reading and understanding the drawings and the description.

The figures may show features that are not recited in the claims to improve the understanding of the claims. These features should be understood as merely optional unless otherwise dictated by context. The individual features of each aspect or embodiment may each be combined with any or all features of other aspects or embodiments.

In the Following Drawings:

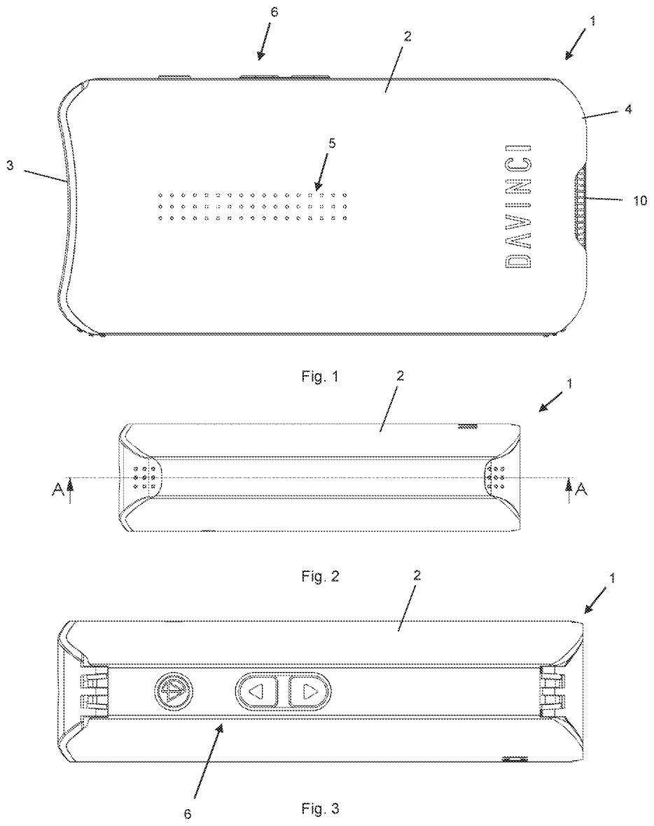

FIGS. 1 to 3 show different perspectives of a vaporizer according to a preferred embodiment of the present invention.

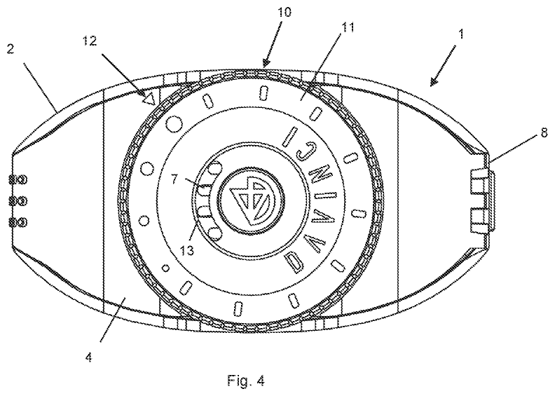

FIG. 4 shows a cross sectional view of the vaporizer FIGS. 1 to 3 along axis A-A:

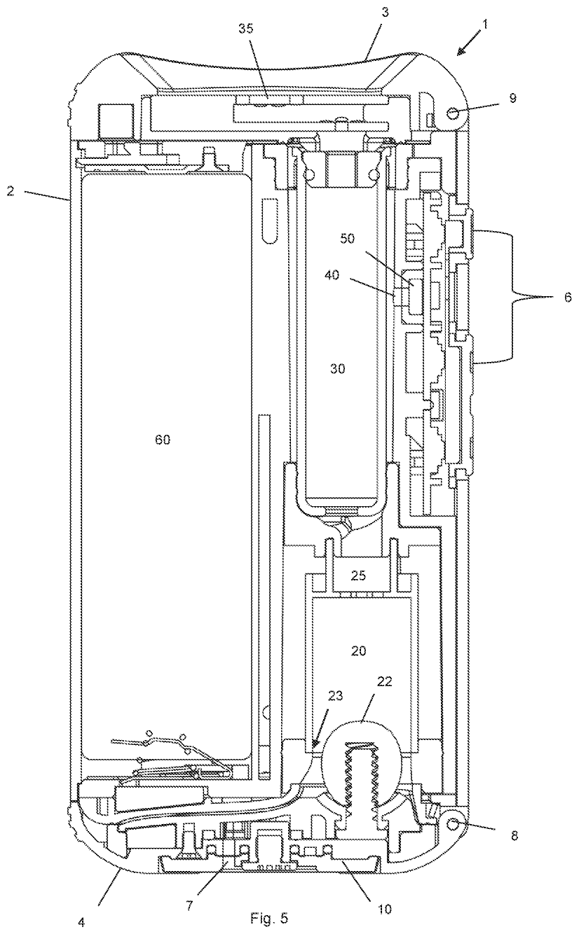

FIG. 5 shows a bottom view of the vaporizer shown in FIGS. 1 to 4.

FIGS. 1 to 3 show a portable loose-leaf material vaporizer 1 according to a preferred embodiment of the present invention. The vaporizer 1 (also referred to herein as the device 1) is for use with loose-leaf cannabis, cannabis concentrates, and other loose-leaf herbs. The device 1 heats the loose-leaf material, generating vapor that contains drug components, then users inhale through the device 1 to consume the vapor.

The vaporizer comprises a housing 2, a mouthpiece 3 and a bottom cap 4 opposite of the mouthpiece 3. The mouthpiece has a shape that conforms to the lips so that users purse their lips against the mouthpiece 3, rather than placing any part of the device 1 into their mouths. This reduces the amount of saliva that is left on the mouthpiece 3 and thus transferred when in a group-sharing setting. A group of LEDs 5 is arranged in an ordered pattern on the housing 2 visibly to the users. The LEDs 5 allow to display information and are also referred to as display LEDs 5. Three buttons 6 are placed on a side of the housing 2 in order to switch the vaporizer 1 on and off, enter and navigate through the menu. In the menu, users may select different modes and input data, as disclosed herein.

The following illustrates by means of a specific example how user input data can be entered in a user interface comprised of buttons 6. When users turn on the vaporizer 1, they have two minutes to enter a so-called “Dosage Control Mode” or DCM. This mode enables the user to input the THC and CBD percentage strength, the size of their bowl, and the status of that bowl—a fresh bowl with fresh herb, a bowl that had been previously heated once, and a bowl that had been previously heated twice. Exiting DCM into regular Smart Path or Precision Temperature modes enables the device to begin calculating and displaying dose via display LEDs 5. Within the two minute window, users are able to access or re-access DCM which also informs them of the inputted values via display LEDs 5. After the 2 minutes window, DCM is no longer accessible. During each inhalation, the button LEDs will light up reacting to the inhalation while the display LEDs 5 will progressively shine more rows of lights over time. After each inhalation, the THC and CBD consumed is shown on the display 5. At the end of the session upon an 8 minute timeout or when the device 1 is turned off before then, the total consumed THC and CBD is displayed.

The vaporizer 1 further comprises an air dial 10 at the bottom cap 4 of the vaporizer 1, which is best seen in FIG. 4. The air dial 10 comprises a rotatable disk 11 and a slot 13 within the disk 11. Depending on the rotational position of the air dial 10, the slot 13 may more or less overlap with the inlet 7 (see FIG. 5) The degree of overlap of the slot 13 with the inlet 7 defines the effective flow cross sectional area and hence the magnitude of resistance exerted by the air dial 10 against the flow through the vaporizer 1. A scale 12 is provided that indicates the magnitude of resistance. The air dial 10 can be pivoted along with the bottom cap 4 around pivot axis 8 to fill the vaporizer 1 with loose-leaf material.

Filling the vaporizer 1 with loose-leaf material is continued to be explained with reference to FIG. 5. The heating chamber 20 is located in the lower half of the vaporizer 1 and is accessible from the bottom of the vaporizer 1. The heating chamber 20 is a hollow tube-shaped oven that receives the loose-leaf material and heats it to a predetermined temperature. By placing the heating chamber 20 at the bottom of the device 1, the temperature of the vapor lowers more as heat is absorbed by the device 1. When the bottom cap 4 is pivoted away, the heating chamber 20 can be accessed from the bottom. After filling the heating chamber 20 with a definite amount of loose-leaf material, the bottom cap 4 is closed, which in turn forces a pearl 22 to protrude into the internal volume of the heating chamber 20. By turning the pearl 22, its height can be adjusted thereby increasing or decreasing the available volume in the heating chamber 20. Changing this can change how much loose-leaf material can be placed into the oven as well as compacting that loose-leaf material to improve the drug extraction.

When inhaling, ambient air flows through the slot 13 of the air dial 10 and through the inlet 7. The air then flows between the annular gap 23 formed between the pearl 22 and the inner surface of the heating chamber 20 into the heating chamber 20, where it mixes with vapor of the loose-leaf material. The mixture of air and vapor exits the heating chamber 20 through heating chamber exit 25, flows then through the flow path 30 to the outlet 35, where the mixture can be withdrawn by pressing the lips on the mouthpiece 3 and inhaling. The flow path 30 directly connects the heating chamber 20 and the mouthpiece 3.

The mouthpiece 3 can be pivoted around a pivot axis 9 away from the housing 2. Thereby, the flow path 30 can be accessed and filed with flavor material. The flow path 30 is therefore also referred to as flavor chamber 30.

A branching 40 branches from the flow path 30 so that the branching 40 is outside of the flow path 30. The branching 40 is a dead-end branch. Thereby the mixture of vapor and air does not flow through the branching 40. A flow detector 50 is arranged in the branching 40. The flow detector 50 is capable to detect whether flow through the flow path 30 occurs.

Elements that stand in contact with vapor and heat, including the heating chamber 20, are preferably made of zirconia ceramic or coated in glass—two materials that are inert and resistant to corrosion and high temperatures. High temperature silicone is preferably used to seal the flow path against leaks.

The vaporizer 1 further comprises a receptacle 60 for receiving a power source (not shown) to power the heating chamber 20 and a processing unit (not shown) as disclosed herein. The processing unit is configured to control temperature of the heating chamber 20.

Attempts to deliver accurate, real-time data regarding THC/CBD dosage are dependent on the ability for the sensors to detect THC/CBD and real-time feedback is dependent on delivery that data during an inhalation. The first issue was found in a lack of available sensors that can directly detect THC/CBD, are very small, simple, and cost-effective. Such sensors have yet to be found. As a result, a direct measurement is unobtainable. This leads to the next best option: an indirect measurement using an air sensor 50. Air sensors are reliable and economical, measuring user inhalation up to 12 seconds. Other required data points, like temperature, time, and cannabis strain data could be acquired through either the device or user input.

Accordingly, the processing unit is configured to estimate a dosage of one or more active agents withdrawn from the vaporizer 1 based on a mathematical model. The mathematical model relates vapor generating time and loose-leaf material properties, and optionally temperature of the heating chamber 20, to the dosage of the one or more active agents withdrawn or to be withdrawn from the vaporizer 1.

The development of the mathematical model for dosage estimation using an empirical approach and a mechanistic approach is described next.

A. Developing an Empirical Mathematical Model for Dosage Estimation

1. Method Development

The consumption process of THC and CBD begins with grinding and homogenizing 0.2-0.5 g of flower into pieces typically 1.2 mm or smaller, then loading that loose-leaf material into the bowl of their device, and finally compacted using a finger or tool. The device is turned on and the bowl heats, beginning decarboxylation or the conversion of THCA and CBDA into THC and CBD respectively. As described herein, temperature and time are assumed to be the primary driving element for decarboxylation and vaporization. The form of THCA and CBDA decarboxylation is taken to be an exponential decay with lower temperatures leading to slower decay and higher temperatures leading to faster decay. Ideal decarboxylation efficiencies of 99.9% and 99.8% for THC and CBD, respectively, is expected within 4 minutes at 410° F., although realistic results will vary due to cooling from inhalations.

As the temperature of the loose-leaf material rises, cannabinoids vaporize into the voids between the loose-leaf particulate. If the user does not inhale, then vapor pressure equilibrium is reached. If the user inhales, then the vaporized cannabinoids are evacuated from the bowl and the heated plant matter is cooled by the in-flowing air. Decarboxylation and vaporization do not stop as a result of this cooling, but their rates do drop considerably the longer the inhalation. Temperature and time are therefore assumed to be the primary driving element for these processes, within reasonable normal use. The form of THCA and CBDA decarboxylation is taken to be an exponential decay with lower temperatures leading to slower decay and higher temperatures leading to faster.

Implementing a dosage algorithm to capture this decarboxylation and vaporization begins in hardware selection. As a starting point, if THC and CBD are present in vapor, then the vapor should be the measurement target. However, direct measurement of vapor is highly improbable given the size and cost constraints of the project. An indirect measurement is therefore the next best option, but that carries its own challenges. Related values such as vapor temperature, flow rate, pressure, and so on are unobtainable as the appropriate sensors are too fragile or too large. One value that is regularly known is the composition of the input material via user input. Another value is the timing as well as duration of each inhalation via a draw or flow sensor. With these two values, a mathematical model can be created to relate input loose-leaf material properties and operating conditions to an output dosage.

Two key assumptions are made: first that all THC and CBD vaporized within the device leaves the device and second that an average loose-leaf cannabis and an average user can be used to create the model. While no cannabis strain nor user adheres perfectly to the average, both assumptions are made to reduce the complexity of the model, while at the same time allowing a good estimate on the dosage. Environmental factors such as air temperature and so on are assumed to be negligible for the average use case.

As the model is based on indirect measurement, a critical component of that model is a reference table created from empirical testing and data. That table would be based on a production rate of THC and CBD. The model would therefore output the THC and CBD produced at a certain time, at a certain temperature, under specific operating conditions. Any amount of THC and CBD produced therefore would also be the amount of THC and CBD consumed. Measuring how much THC and CBD is produced can be conducted by measuring the amount of THC and CBD lost within heated loose-leaf material. An additional benefit of measuring heated material is that the effects from decarboxylation are inherently included within the data.

Other considerations such as the decarboxylation efficiency, distribution of THC and CBD in vapor vs. residue, and effects due to an airflow control valve (air dial) built into the device would also be explored, though ultimately decided to be negligible.

The Initial Algorithm Follows:

[A]×[B]×[C]×[D]×[E]=[THCand CBD produced] (1)

where [A] is the mass of the loose-leaf material, [B] is the percentage concentration of THC and CBD present, [C] is the reference table production rate and based on temperature and time conditions, [D] is the inhalation duration, [E] represents any other effects that might be discovered during testing.

Additionally, only [C] is a reference-based value whereas all other variables are either fixed or measured by sensors.

2. Hypothesis

There were 3 Hypotheses Tested:

#1: The amount of THC and CBD lost within heated loose-leaf material is affected by temperature, time, and inhalation duration. Changing those variables will change the lost amount, thus the produced amount, thus the consumed amount.

#2: The amount of THC and CBD lost within heated loose-leaf material is affected by the time it spends being heated, but not inhaled. Changing that variable will change the amount available for consumption.

#3: The amount of THC and CBD lost within heated loose-leaf material is affected by flow rate and pressure caused by the ‘air dial’ feature. Changing the air dial setting will change the flow rate and pressure, thus changing the list amount.

These hypotheses would be tested in 3 phases: a preliminary phase using averaged human parameters to explore hypothesis #3 and provide initial data for hypothesis #1 and #2, a primary phase using averaged human parameters to explore hypothesis #1 and #2, and a final phase using real human subjects to adjust the reference table.

3. Testing and Data Analysis

3.1 Procedures

For Phase 1 and Phase 2, the procedures focused on preparing samples of heated loose-leaf material for the lab to test. Phase 3 focused on surveying users and then adjusting numerical values to better reflect user feedback.

3.2 Materials

The materials used for testing can be categorized as cannabis, equipment, and sample containment. In each phase of testing, one batch of cannabis flower (sativa, 20-30% THC, 0-1% CBD) was purchased at local dispensaries and used for testing. Equipment consisted of a grinder, pump, tubing, and other hardware needed to expedite sample preparation during Phase 1 and Phase 2. Sample containment consisted or containers used to house the cannabis flower as well as transport heated loose-leaf material to the testing lab.

3.3 Testing Phase 1: Preliminary

The goals of preliminary testing were to determine the parameters of an average user, troubleshoot the procedures, test hypothesis #3, and obtain initial data for hypothesis #1 and #2 with the intent of condensing the scope of the primary testing phase. Early forecasts placed test quantities of 150-200 samples in the primary testing phase alone, which would be a significant cost for the project. Each of these goals were fulfilled, though many opportunities for improvement are present—See Discussion section.

The average user was determined through an informal, internal survey. 6 test subjects were surveyed where each would be given an IQ device, cannabis flower, and then asked to “vape” as they would normally across a light, medium, and heavy inhalation. The inhalations and delay between inhalations were observed and timed. Additionally, each test subject self-rated their THC tolerance. The IQ device was chosen. The results of this survey are listed below in Table 1.

TABLE 1
Internal Survey on the Determination of Standard User Parameters

| | | | Inhalation Parameters | | | | |
|---|---|---|---|---|---|---|---|
| | | | Light | | | Average | Delay |
| | Flower | Toler- | (sec- | | | (sec- | (sec- |
| User | Used (g) | ance | onds) | Medium | Heavy | onds) | onds) |
| LT | 0.3 | Light | 2.17 | 2.05 | 2.82 | 2.23 | 18.96 |
| RM | 0.4 | Medium | 7.74 | 6.93 | 10.01 | 8.23 | 5.69 |
| HM | 0.3 | Medium | 2.62 | 2.09 | 5.44 | 3.38 | 10.43 |
| MB | 0.4 | Heavy | 2.23 | 3.93 | 8.74 | 4.97 | 8.66 |
| PM | 0.3 | Heavy | 3.72 | 7.45 | 16.19 | 9.12 | N/A |
| JB | 0.4 | Heavy | 6.21 | 6.19 | 8.84 | 7.08 | 25.88 |
| | | | | | Average | 5.85 | 13.92 |

From Table 1, the average inhalation time was rounded from 5.85 seconds to 6 seconds and the average delay between inhalations was rounded from 13.92 seconds to 14 seconds to result in a draw frequency of 3 draws per minute. Using this data, 5 samples were prepared and submitted to the lab. The condition as well as the lab result for each of the samples are listed below in Table 2.

TABLE 2
Comparison of THC and CBD Loss between Air Dial Settings and
Runtimes - Preliminary Test Results Obtained via LCMS Testing

| | Sample | | | | |
|---|---|---|---|---|---|
| | | 25% Air | 100% Air | 25% Air | 100% Air |
| | | Dial | Dial | Dial | Dial |
| | | 8 min | 8 min | 16 min | 16 min |
| Cannabinoid* | Control | runtime | runtime | runtime | runtime |
| Total | 19.620 | 17.249 | 16.714 | 14.282 | 15.910 |
| THC (%) | | | | | |
| THCA** | 21.066 | 8.266 | 6.634 | 4.872 | 5.749 |
| Δ9-THC | 1.145 | 10.000 | 10.896 | 10.009 | 10.868 |
| CBDA** | 1.635 | 1.635 | <LOQ | <LOQ | <LOQ |
| CBD | <LOQ | <LOQ | <LOQ | <LOQ | <LOQ |

*Other cannabinoids such as Δ8-THC, CBN, and so on are omitted as they are not of interest for this report and also tested below the Limit of Quantitation (<LOQ).
**THCA and CBDA both mostly convert into Δ9-THC and CBD, respectively.

Due to the bare CBD data, only the THC data was usable. By determining the Total THC loss over time and across Air Dial settings, the effect of the Air Dial can be determined. Since the difference between settings was about +0.4%/−0.5% and there being such a small sample size (sample size=5), this result was determined to be unreliable. However, it was decided to be negligible and thus removed from consideration for further testing since the little data present showed a nearly insignificant difference between settings. This decision disproved hypothesis #3.

By visualizing the Total THC loss overtime, as well as the Total THC produced, the beginnings of the mathematical model are created—the model originates from THC loss in the heated loose-leaf material, equates to the THC gain in the vapor, and ultimately the slope of that gain is found to determine production rate.

3.4 Testing Phase 2: Primary

The goals of primary testing are to test hypothesis #1 and #2, thereby creating the reference table and explore the difference between inhalation frequencies. Following the preliminary testing, the test quantity required for the primary phase was reduced from an estimated 150-200 samples down to 66 samples for the reference table. However, only 43 of the 66 samples could be tested as the remainder was reallocated to exploring other considerations. The empty data points between the test samples were interpolated. The goals of this section can also be considered fulfilled, but one step was made that requires further exploration—See Discussion section.

A total of 59 samples were submitted to the testing lab. These samples were prepared across the range of temperatures allowed by the IQ device—from a room temperature of 70° F. up to a maximum of 430° F.—and across the range of times most commonly used—up to 24 minutes of continuous use. Temperature and usage time are inversely related where the hotter a user vapes, the less time they will vape for as vapor flavor will rapidly begin to deteriorate. The data points relevant to the reference table are listed below in Table 3.

TABLE 3
THC Loss Over Time at a Range of Temperatures

| | Temperature | | | | | | |
|---|---|---|---|---|---|---|---|
| | 70 | | | | | | |
| Time | (° F.) | 310 | 350 | 370 | 390 | 410 | 430 |
| 0 | 0.2094 | Same as control** | | | | | |
| (sec- | | | | | | | |
| onds) | | | | | | | |
| 60 | Same as | 0.22414 | 0.23383 | 0.23065 | 0.23357 | 0.23266 | 0.22180 |
| 120 | control | * | * | * | 0.21442 | 0.23846 | 0.23583 |
| 240 | | 0.20373 | 0.22124 | 0.22701 | 0.20137 | 0.19082 | 0.19059 |
| 360 | | * | * | * | 0.18504 | 0.18337 | 0.17198 |
| 480 | | 0.21378 | 0.18400 | 0.20764 | 0.18675 | 0.16611 | 0.16050 |
| 600 | | * | * | * | 0.16460 | 0.16913 | 0.15810 |
| 720 | | * | * | * | 0.17318 | * | * |
| 840 | | 0.22513 | 0.17915 | 0.19567 | 0.16993 | 0.18206 | 0.15920 |
| 960 | | * | * | * | 0.17888 | * | * |
| 1200 | | * | * | * | 0.16349 | * | * |
| 1400 | | 0.20492 | 0.17749 | 0.17398 | 0.17326 | 0.14072 | 0.15525 |

*Data points marked with (*) were omitted from testing due to tack of available budget. These points were chosen based on which temperatures users most commonly used and disused.
**“Same as control” indicates the data point for 0 seconds/70° F.

In order to process the values into usable data, some adjustments are required to account for variations in sample size and then generalizations and smoothing. The result represents the THC Loss, in percentage, for any input cannabis loose-leaf material if the initial conditions are close to that of the average user. Outlier conditions may exist, but this model provides a starting point for modifications to the model in later design iterations. Inverting the data converts the values from THC loss in heated loose-leaf material to THC gain in vapor. The results represent the THC gain of vapor, in percentage, for any input cannabis loose-leaf material if the initial conditions are close to that of the average users and their flower. An assumption is made here in that THC gain will be the same for any amount of THC present and available in flower whether 0.5% or 30%.

Two additions were made that revised the size of the reference table's data ranges, namely:
(i) 11 time ranges increased to 12 time ranges to give greater clarity to the first minute of heating.
(ii) 6 temperature ranges increased to 7 temperature ranges to give greater clarity to the 350-430° F. temperature ranges where most users would be most active.

The blank values where interpolated, and the slope found. An arbitrary coefficient of 30 was multiplied as well to allow to scale the entire table up or down based on user feedback later. The adjusted values are listed below in Table 4. The unit of this table can be expressed as either % THC per second or second\hat{ }−1.

TABLE 4
THC Production Rate

| | | Temperature Range | | | | | | |
|---|---|---|---|---|---|---|---|---|
| | | Floor | | | | | | |
| | | 250 | 290 | 310 | 350 | 370 | 390 | 410 |
| | | Ceiling | | | | | | |
| Time Range | | 289 | 309 | 349 | 369 | 389 | 409 | 430 |
| 0 | 29 | 0.00050 | 0.00071 | 0.00420 | 0.00556 | 0.00733 | 0.00779 | 0.00864 |
| 30 | 59 | 0.00070 | 0.00222 | 0.00720 | 0.01111 | 0.01476 | 0.01557 | 0.01727 |
| 60 | 119 | 0.00222 | 0.00385 | 0.00702 | 0.00966 | 0.01112 | 0.01200 | 0.01300 |
| 120 | 239 | 0.00235 | 0.00354 | 0.00599 | 0.00771 | 0.00811 | 0.00852 | 0.00900 |
| 240 | 359 | 0.00221 | 0.00279 | 0.00435 | 0.00501 | 0.00522 | 0.00622 | 0.00700 |
| 360 | 479 | 0.00118 | 0.00245 | 0.00350 | 0.00391 | 0.00420 | 0.00437 | 0.00500 |
| 480 | 599 | 0.00111 | 0.00235 | 0.00332 | 0.00301 | 0.00388 | 0.00333 | 0.00388 |
| 600 | 719 | 0.00105 | 0.00210 | 0.00249 | 0.00255 | 0.00266 | 0.00298 | 0.00288 |
| 720 | 839 | 0.00090 | 0.00151 | 0.00179 | 0.00205 | 0.00225 | 0.00267 | 0.00211 |
| 840 | 959 | 0.00083 | 0.00137 | 0.00166 | 0.00176 | 0.00201 | 0.00232 | 0.00205 |
| 960 | 1199 | 0.00063 | 0.00113 | 0.00126 | 0.00112 | 0.00137 | 0.00157 | 0.00198 |
| 1200 | 1440 | 0.00053 | 0.00101 | 0.00075 | 0.00085 | 0.00095 | 0.00101 | 0.00155 |

CBD production rate can be found with the same process and tested for concurrently with THC. Both flower strains purchased at the dispensary for Phase 1 and Phase 2 testing listed 0.30-0.50% CBD which would have been detectable even after heating, except the control samples tested at 0.00% CBD. As a temporary measure, the CBD production rate table was decided to be adapted from the THC production rate table and then retested for later.

The adaptation from THC to CBD production rate consisted of shifting columns by 5.8%: for the temperature ranges below the boiling point of CBD, they would be shifted down by 5.8% which those ranges above would be shifted up by 5.8%. This assumes that CBD production rate is less active at cooler temperatures and more active at higher temperatures on either side of its boiling points. The result of adapting THC to CBD production rates is listed below in Table 5.

TABLE 5
CBD Production Rate

| | | Temperature Range | | | | | | |
|---|---|---|---|---|---|---|---|---|
| | | Floor | | | | | | |
| | | 250 | 290 | 310 | 350 | 370 | 390 | 410 |
| | | Ceiling | | | | | | |
| Time Range | | 289 | 309 | 349 | 369 | 389 | 409 | 430 |
| 0 | 29 | 0.00047 | 0.00067 | 0.00396 | 0.00588 | 0.00781 | 0.00824 | 0.00913 |
| 30 | 59 | 0.00066 | 0.00209 | 0.00678 | 0.01175 | 0.01561 | 0.01647 | 0.01827 |
| 60 | 119 | 0.00209 | 0.00363 | 0.00661 | 0.01022 | 0.01176 | 0.01269 | 0.01375 |
| 120 | 239 | 0.00221 | 0.00334 | 0.00564 | 0.00616 | 0.00858 | 0.00901 | 0.00952 |
| 240 | 359 | 0.00208 | 0.00263 | 0.00410 | 0.00530 | 0.00552 | 0.00658 | 0.00741 |
| 360 | 479 | 0.00111 | 0.00231 | 0.00330 | 0.00413 | 0.00444 | 0.00463 | 0.00529 |
| 480 | 599 | 0.00105 | 0.00221 | 0.00313 | 0.00318 | 0.00410 | 0.00352 | 0.00410 |
| 600 | 719 | 0.00099 | 0.00198 | 0.00235 | 0.00270 | 0.00281 | 0.00315 | 0.00305 |
| 720 | 839 | 0.00085 | 0.00142 | 0.00169 | 0.00217 | 0.00238 | 0.00282 | 0.00223 |
| 840 | 959 | 0.00078 | 0.00129 | 0.00156 | 0.00186 | 0.00213 | 0.00245 | 0.00217 |
| 960 | 1199 | 0.00059 | 0.00106 | 0.00119 | 0.00118 | 0.00145 | 0.00166 | 0.00209 |
| 1200 | 1440 | 0.00050 | 0.00095 | 0.00071 | 0.00090 | 0.00100 | 0.00107 | 0.00164 |

Two additional variables were investigated: “Draw Capture” and “Resting Loss”. Draw capture is an application of hypothesis #1 to the inhalation itself, where the production rate will change at each second of the inhalation. Resting loss represents the effect of hypothesis #2, in which prolonged heating can cause THC to either escape the device or decompose into other compounds. Resting loss is only significant to a degree in longer duration use sessions as a majority of the initial few minutes of every session is spent decarboxylating THCA into THC. Each of these variables were investigated in the same way as THC production rate, but the samples were prepared differently.

For draw capture, the timing of the inhalations used during sample preparation was changed from a 6-second inhalation every 20 seconds to 5/10/17-second inhalations every 30 seconds. These samples were tested at 390° F. The loss was compared between time and then interpolated out to its own reference table. This draw capture loss rate is listed below in Table 6.

TABLE 6
Draw Capture Rate

| Time Range | | |
|---|---|---|
| Floor | Ceiling | Modifier |
| 0 | 0.9 | 1.00000 |
| 1 | 1.9 | 1.00000 |
| 2 | 2.9 | 0.94393 |
| 3 | 3.9 | 0.88785 |
| 4 | 4.9 | 0.83178 |
| 5 | 5.9 | 0.77571 |
| 6 | 6.9 | 0.71963 |
| 7 | 7.9 | 0.66356 |
| 8 | 8.9 | 0.60748 |
| 9 | 9.9 | 0.55141 |
| 10 | 16.9 | 0.49534 |
| 17 | 99* | 0.10282 |

For resting loss, loose-leaf material was loaded into the IQ device and baked for an amount of time. These samples were tested at 390° F. The results were evaluated and when the loss rate was used where the rate change reached closer to steady state. This resting loss rate is listed below in Table 7.

TABLE 7
Resting Loss

| Time Range | | |
|---|---|---|
| Floor | Ceiling | Modifier |
| 0 | 14 | 1.00000 |
| 15 | 29 | 0.99286 |
| 30 | 44 | 0.98572 |
| 45 | 59 | 0.97858 |
| 60 | 119 | 0.97144 |
| 120 | 239 | 0.94287 |
| 240 | 480 | 0.88575 |
| 481 | 960 | 0.77150 |
| 961 | 1440 | 0.54300 |

The results in Table 4 through Table 7 conclude Phase 2 testing and modifications are made to Equation 1 such that the modified algorithm follows:

[A]×[B]×[C]×[D]×[E]×[F]×[G]=[THCand CBD produced per sec.] (2)

Where [A] is the mass of the loose-leaf material, [B] is the percentage concentration of THC and CBD present. [C] is the reference table production rate and based on temperature and time conditions, [D] is the inhalation duration of 1 second, [E] is the reference table draw capture modifier, [F] is the reference resting loss modifier, [G] is a reference “depletion state” modifier.

The depletion state modifier is a failsafe measure to prevent the THC and CBD produced per session from exceeding the theoretical maximum THC and CBD producible, which is simple and easy to calculate. For example, a user has 0.2 g of cannabis flower at 20% THC, yielding 40 mg of THC. If the session THC is 50 mg when only 40 mg is available, then end users will cast doubt onto the validity of the mathematical model. This modifier is rarely used for loose-leaf material—it is more prevalent with concentrates. When the calculated session dose is less than the theoretical maximum dose, this value is set equal to 1. When the dose exceeds maximum, this value is set equal to 0.01.

The firmware and/or mobile application would handle how additional values are calculated and displayed such as THC and CBD produced per inhalation. THC and CBD produced per session, and other historical data points.

3.5 Testing Phase 3: Final

The goal of final testing is to adjust the reference tables using real human test subjects.

A total of 5 subjects were used in this testing phase. Each subject was asked to “vape” at regular intervals (5 second inhalations every 30 seconds). The dose per inhalation from Equation 2 is recorded and then compared to both user expectation and vapor density expelled after each inhalation. While not entirely scientifically reasonable, the market holds the perception that denser vapor and larger vapor clouds equates to a higher high.

As a result of the adjustment, Table 4 through Table 6 are revised to Table 7 through Table 10 below.

TABLE 7
THC Production Rate

| | | Temperature Range | | | | | | |
|---|---|---|---|---|---|---|---|---|
| | | Floor | | | | | | |
| | | 250 | 290 | 310 | 350 | 370 | 390 | 410 |
| | | Ceiling | | | | | | |
| Time Range | | 289 | 309 | 349 | 369 | 389 | 409 | 430 |
| 0 | 29 | 0.00008 | 0.00012 | 0.00073 | 0.00111 | 0.00253 | 0.00350 | 0.00475 |
| 30 | 59 | 0.00023 | 0.00078 | 0.00288 | 0.00556 | 0.00959 | 0.01246 | 0.01468 |
| 60 | 119 | 0.00100 | 0.00183 | 0.00351 | 0.00676 | 0.00873 | 0.01020 | 0.01170 |
| 120 | 239 | 0.00118 | 0.00195 | 0.00359 | 0.00694 | 0.00750 | 0.00809 | 0.00378 |
| 240 | 359 | 0.00166 | 0.00223 | 0.00370 | 0.00501 | 0.00522 | 0.00622 | 0.00700 |
| 360 | 479 | 0.00104 | 0.00221 | 0.00333 | 0.00391 | 0.00420 | 0.00437 | 0.00500 |
| 480 | 599 | 0.00105 | 0.00229 | 0.00332 | 0.00301 | 0.00388 | 0.00333 | 0.00388 |
| 600 | 719 | 0.00105 | 0.00210 | 0.00249 | 0.00255 | 0.00266 | 0.00298 | 0.00288 |
| 720 | 839 | 0.00090 | 0.00151 | 0.00188 | 0.00205 | 0.00225 | 0.00267 | 0.00211 |
| 840 | 959 | 0.00083 | 0.00140 | 0.00174 | 0.00176 | 0.00201 | 0.00232 | 0.00205 |
| 960 | 1199 | 0.00064 | 0.00119 | 0.00139 | 0.00112 | 0.00137 | 0.00157 | 0.00198 |
| 1200 | 1440 | 0.00056 | 0.00107 | 0.00075 | 0.00085 | 0.00095 | 0.00101 | 0.00155 |

TABLE 8
CBD Production Rate

| | | Temperature Range | | | | | | |
|---|---|---|---|---|---|---|---|---|
| | | Floor | | | | | | |
| | | 250 | 290 | 310 | 350 | 370 | 390 | 410 |
| | | Ceiling | | | | | | |
| Time Range | | 289 | 309 | 349 | 369 | 389 | 409 | 430 |
| 0 | 29 | 0.00007 | 0.00012 | 0.00073 | 0.00118 | 0.00273 | 0.00371 | 0.00502 |
| 30 | 59 | 0.00022 | 0.00073 | 0.00271 | 0.00588 | 0.01015 | 0.01318 | 0.01553 |
| 60 | 119 | 0.00094 | 0.00172 | 0.00331 | 0.00715 | 0.00923 | 0.01079 | 0.01238 |
| 120 | 239 | 0.00111 | 0.00183 | 0.00339 | 0.00734 | 0.00794 | 0.00856 | 0.00928 |
| 240 | 359 | 0.00156 | 0.00210 | 0.00348 | 0.00530 | 0.00552 | 0.00658 | 0.00741 |
| 360 | 479 | 0.00098 | 0.00208 | 0.00313 | 0.00413 | 0.00444 | 0.00463 | 0.00529 |
| 480 | 599 | 0.00099 | 0.00216 | 0.00313 | 0.00313 | 0.00410 | 0.00352 | 0.00410 |
| 600 | 719 | 0.00099 | 0.00198 | 0.00235 | 0.00270 | 0.00281 | 0.00315 | 0.00305 |
| 720 | 839 | 0.00085 | 0.00142 | 0.00177 | 0.00217 | 0.00238 | 0.00282 | 0.00223 |
| 840 | 959 | 0.00078 | 0.00132 | 0.00164 | 0.00186 | 0.00213 | 0.00245 | 0.00217 |
| 960 | 1199 | 0.00061 | 0.00112 | 0.00131 | 0.00113 | 0.00145 | 0.00166 | 0.00209 |
| 1200 | 1440 | 0.00052 | 0.00100 | 0.00071 | 0.00090 | 0.00100 | 0.00107 | 0.00164 |

TABLE 3
Draw Capture Rate

| Time Range | | |
|---|---|---|
| Floor | Ceiling | Modifier |
| 0 | 0.9* | 1.00000 |
| 1 | 1.9 | 1.00000 |
| 2 | 2.9 | 0.94393 |
| 3 | 3.9 | 0.88785 |
| 4 | 4.9 | 0.83178 |
| 5 | 5.9 | 0.77571 |
| 6 | 6.9 | 0.71963 |
| 7 | 7.9 | 0.66356 |
| 8 | 8.9 | 0.60748 |
| 9 | 9.9 | 0.55141 |
| 10 | 16.9* | 0.49534 |
| 17 | 99* | 0.10282 |

*Part of these time ranges are invalid due to the 12-second limitation of the air sensor. They have no impact on the result

TABLE 10
Resting Loss

| Time Range | | |
|---|---|---|
| Floor | Ceiling | Modifier |
| 0 | 14 | 1.00000 |
| 15 | 29 | 0.99286 |
| 30 | 44 | 0.98572 |
| 45 | 59 | 0.97858 |
| 60 | 119 | 0.97144 |
| 120 | 239 | 0.94287 |
| 240 | 480 | 0.88575 |
| 481 | 960 | 0.77150 |
| 961 | 1440 | 0.54300 |

3.6 Mathematical Model

Phase 3 testing did not influence the structure of the mathematical model, only the numerical values in the reference tables. As a result, Equation (2) stands as the most current algorithm, where [A] is the mass of the loose-leaf material, [B] is the percentage concentration of THC and CBD present, [C] is the reference table production rate and based on temperature and time conditions (See Table 7 for THC and Table 8 for CBD), [D] is the inhalation duration of 1 second, [E] is the reference table draw capture modifier (See Table 9), [F] is the reference resting loss modifier (See Table 10), [G] is a reference depletion state modifier.

4. Conclusion and Discussion

This report represents an approach into the determination of precision dosing for loose-leaf cannabis. Other devices that claim to dose use pre-measured containers of cannabis oil, limiting their heating mechanisms by an approximation of mass loss and thus cannabinoid consumption. By departing from that dosing method, the mathematical model gives a framework to build from that is flexible for a variety of use cases and economical in the hardware it uses. The behavior of THC and CBD confirmed expectations. Cooler temperatures led to both slower decarboxylation and vaporization while hotter temperatures led to faster activity.

The results of Phase 1 and Phase 2 testing were particularly revealing, in that about 40-50% of THC was removed from the loose-leaf material across a 24-minute time range. Later surveys and observations during Phase 3 indicated that an inhalation frequency of 3 inhalations per minute was much higher than normal for the average use case, and high even for a shared setting. As the temperature of the loose-leaf material reached steady-state equilibrium with the consistent inhalations, the decarboxylation of THCA slowed significantly with about 4% THCA remaining unconverted and about 12.5% THC available, but unvaporized due to the constant cooling. A variation of the testing would look like repeating the tests for a single user and a group of users and changing the inhalation frequency to be more reasonable. Overall, the model follows behavior set by previous studies.

B. Developing a Mechanistic Mathematical Model for Dosage Estimation

1. Decarboxylation of Medical Cannabis

THCA is found in abundance in growing and harvested cannabis and is a biosynthetic precursor of THC. THCA is converted into THC when heat is added (known as decarboxylation or activation). The conversion is a naturally occurring chemical reaction, the rate of which is greatly increased at higher temperatures. The released carboxylic acid group is converted to CO2 gas during the process.

THC activation is a mathematical calculation to determine what percentage of the combined THCA & THC molecules is in the activated THC form. To do this, we use the following equation:

THC Activation=THC value/(THC value+THCA value)*100% (3)

Decarboxylation of THCA into THC starts at 90° C. At 100° C. it takes 3 hours to convert THCA fully into THC. At 160° C. it takes 10 Minutes to convert THCA fully into THC. At 200° C. it takes seconds to convert THCA fully into THC.

Starting from 157° C. THC evaporates. The point of CBD is 180° C. Because of the small quantities of THC and CBD that evaporate, it can be assumed, that the already decarboxylated amounts of THC and CBD immediately evaporate and that any newly produced THC and CBD also immediately are available in the gaseous phase. Evaporation stops immediately, once the temperature is reduced to values below the boiling points.

The volume of evaporated THC and CBD needs to be defined. If 1 gram of herbal cannabis type bediol with 6.3% total THC and 8% total CBD is decarboxylated and evaporated, 63 mg THC and 80 mg CBD are produced. The molar mass is 314.469 g/mol for THC and 314.464 g/mol for CBD, therefore, if can be assumed that both components have the same molar mass of 314.5 g/mol.

The total of 0.143 g of THC and CBD correspond to 0.000455 mol of active components. If we assume that the vaporized THC and CBD can be treated as ideal gas, one mol would be a volume of 22,4 litres. Therefore, the THC and CBD contained in 1 gram of BEDIOL corresponds to 10.2 cm².

During decarboxylation and vaporization typically only 25% of active components are vaporized for one draw (four draws per charge). If the total volume of the vapor path is larger than 2.5 cm² it could be assumed that no active components leaves the vapor path.

Some articles report, that only 67% THCA was converted to THC, because of side reactions (THCA and THC to CBNA and CBN). It is also reported, that the total mass of THCA/THC or CBDA/CBD is reduced after decarboxylation. Degradation of THC to CBN starts at 85° C. E.g. optimal conversion of THCA to THC was observed for a temperature of 150° C. For higher temperatures side products like CBN (Cannabinol) result and conversion rate is lowered. According to a further report, during smoking a joint, most THC is destroyed and the consumer can take up only about 30% of the active substance.

The total mass of the products (e.g. THCA & THC or CBDA & CBD) after such a decarboxylation reaction is reduced compared to the initial mass. The reduction was reported to be 7.94% for THCA/THC and 18.05% and 13.75% for CBDA/CBD and extracts and pure standard material, respectively.

The relationship between the rate of the decarboxylation reaction d[C]/dt concentration of acidic cannabinoids [C] can be expressed by Eq. 3 or the alternative Eq. 4:

D[C]/dt=−k*[C]

In([C]₀/[C]ₜ)=k*t (4)

where k presents the rate constant, and [C]₀ and [C]ₜ are the concentrations of reactants at time 0 and t min. respectively.

The activation energy, E_{A}, which indicates the minimum energy for the reaction to occur, can be determined from the temperature dependence of the rate constant by the so-called Arrhenius equation, Eq. 5:

In k=In k₀−E_{A}/(R*T)

where k₀ is the frequency factor and R is the gas constant.

For THCA, the following values have been published:

E_{A}=84.8 kJ/mol with k₀=3.7*10⁸ sec⁻¹. Experiments were reproduced by others resulting in the values E_{A}=88 kJ/mol with k₀=8.7*10⁸ sec⁻¹. E

For CBDA corresponding experimental values for the rate constant can be found. This allows calculating the amount of acidic cannabinoids that was already decarboxylated.

TABLE 11

| | Rate constants k (times 10³) [sec⁻¹] | | | Activation Energy |
|---|---|---|---|---|
| Reactant | 80° C. | 95° C. | 110° C. | E_{A} [kJ/mol] |
| THCA | 0.18 | 0.66 | 1.83 | 88 |
| CBDA | 0.05 | 0.27 | 0.83 | 112 |

The rate constant of CBDA is nearly always approximately 50% of the rate constant of THCA. Using Arrhenius' law, the following values for the activation energy results E_{A}=98.51 kJ/mol and k₀=2.24*10¹⁰ sec⁻¹.

These derived values of E_{A} and k₀ can be used to calculate the k-values for higher temperatures, which allows to calculate the conversion of THCA to THC and CBDA to CBD at higher temperatures as typically used in vaporizers.

TABLE 12

| | Rate constants k (times 10³) [sec⁻¹] | | | | | | | | |
|---|---|---|---|---|---|---|---|---|---|
| Reactant | 80° C. | 95° C. | 110° C. | 130° C. | 150° C. | 170° C. | 190° C. | 210° C. | 230° C. |
| THCA | 0.18 | 0.66 | 1.83 | 5.55 | 18.78 | 56.90 | 156.63 | 396.50 | 932.31 |
| CBDA | 0.05 | 0.27 | 0.83 | 3.83 | 15.35 | 54.34 | 172.44 | 497.34 | 1318.58 |

A value of k=693 sec⁻¹ means that 50% of the acidic cannabinoids is decarboxylated within one second, which is the case for temperatures around 210° C. A value of k=10 sec⁻¹ means that only 1% of the acidic cannabinoids is decarboxylated within one second.

To result in a decarboxylation of 25% of the acidic cannabinoids (e.g. to release the amount of active components for one draw) it would be sufficient to heat the herbal cannabis up to 170° C. where k-values of approx. 55 results. With such a k-value it takes only 5 seconds to convert 25% of the acidic components. Since the temperature is above the boiling point of THC and CBD it could be assumed, that after 5 seconds the 25% of contained active components are ready to be delivered to the patient and the herbal cannabis needs to be cooled down immediately.

If the same temperature would be used prior to the next draws, the time when the next 25% of active components are ready for the next draw increases, since the initial amount of acidic components is already reduced (to 75, 50, 25%). The time prior to the second draw increases to 7.5 seconds, for the third draw to 12.5 seconds and for the fourth draw to >40 seconds.

TABLE 13
Calculated time for delivery of approx. equal amounts of active
components for each draw at a constant temperature.

| Constant temperature | Temperature [° C.] | Time to be ready [sec] |
|---|---|---|
| Draw 1 | 170 | 5 |
| Draw 2 | 170 | 7.5 |
| Draw 3 | 170 | 12.5 |
| Draw 4 | 170 | >40 |

Alternatively, the temperature used prior to the second, third and fourth draw can be increased to result in the same time to be ready. The following k-values would be needed to convert 25% or active components within 5 seconds, 55, 80, 140 and 1000, which corresponds to the following temperatures: 170° C., 176.5° C. 187.7° C. and 230° C.

TABLE 14
Target temperatures for a temperature profile delivering
approx. equal amounts of active components for each draw.

| Constant time to be ready | k-value [sec⁻¹] | Temperature [° C.] |
|---|---|---|
| Draw 1 | 55 | 170 |
| Draw 2 | 80 | 176.5 |
| Draw 3 | 140 | 187.7 |
| Draw 4 | 1000 | 230 |

Such temperatures would be the starting points for the temperature profiles resulting in the delivery of equal amounts of active components for each draw.

The calculated k-values were used to calculate the THCA/CBDA and THC/CBD content resulting from an initial, not optimized temperature profile. The temperature was increased from 170° C., 176° C. 186° C. and 230° C. for the four drawing cycles. The raise time (heating up) was assumed to be 1° C. per 0.1 seconds, the cooling caused by the fresh air going through the heating chamber (turned off) was assumed to be 5° C. per 0.1 seconds. The resulting k-values for the decarboxylation of THCA and CBDA was calculated. The THCA and CBDA content was reduced by approx. 25% for each draw cycle. The produced THC and CBD was calculated and was increasing by approx. 25% prior to each draw. When the temperature was increased above the boiling point of THC (157° C.) and CBD (180° C.) the produced THC and CBD was released into the air and can be consumed by the patient via a draw.

Since the boiling point of CBD is above the maximal temperatures of the first two draws, all CBD is still bound within the herbal cannabis. During the heating phase of the third draw (up to 186° C.) the boiling point of CBD is reached and all bound CBD is released. Therefore, the amount of CBD in the four draws is different: the CBD content in the first two draws is close to zero, while the third draw contains nearly 75% of the available CBD and the fourth draw contains 25% of CBD. All four draws contain approx. 25% of THC.

Several assumptions and simplifications were made, which need to be validated and tested for a specific vaporizer. E.g. the temperature raise time needs to be adapted to the configuration of the heating chamber and the temperature drop caused by a draw needs to be evaluated by a corresponding experiment. Further, the calculated and used k-values need to be validated by appropriate experiments.

2. Mathematical Model

In a next step, a simulation model using these rate constants k is built, which enables to calculate the total amount of THC and CBD after a certain time at a given temperature. Such a model ideally considers the following points:
(i) Because of continuous temperature increase (e.g. from room temperature to target temperature) the integral over time of the heat mediated decarboxylation should be evaluated, for each temperature the calculated k-value needs to be used (see above).
(ii) Because of the inhomogeneous temperature profile within the heated herbal cannabis, also an integration over space could be evaluated.
(iii) Degradation of THC and CBD within the herbal cannabis needs to be considered by limiting the total amount of converted THC to 70%
(iv) The actual vaporization of THC/CBD starts at 157° C./180° C. and makes the active components immediately available to be inhaled.

Such a model with its various assumptions (immediate vaporization, extrapolated k-values etc.) is then validated and, if necessary, adjusted by real measurements.

1.-15. (canceled)

16. Portable loose-leaf material vaporizer comprising:
a heating chamber for housing loose leaf material and vaporizing one or more active agents from the loose-leaf material,
a mouthpiece for withdrawal of the one or more active agents vaporized from the loose-leaf material, and
a processing unit,
wherein the processing unit is configured to control temperature of the heating chamber and to estimate a dosage of the one or more active agents withdrawn from the vaporizer based on a mathematical model,
wherein the mathematical model relates vapor generating time and loose-leaf material properties to the dosage of the one or more active agents withdrawn or to be withdrawn from the vaporizer.

17. The portable loose-leaf material vaporizer of claim 1, wherein the estimation is performed continuously.

18. The portable loose-leaf material vaporizer of claim 1, wherein the processing unit is configured to determine, based on the mathematical model, one or more of the following:
(i) the vapor generating time until a predetermined dosage is available for withdrawal from the vaporizer and/or
(ii) the temperature or temperature profile of the heating chamber so that a predetermined dosage is available within a predetermined vapor generating time for withdrawal from the vaporizer and/or
(iii) the number of draws until a predetermined dosage is withdrawn from the vaporizer.

19. The portable loose-leaf material vaporizer of claim 1, wherein the processing unit is configured to stop heating of the heating chamber and/or start cooling of the heating chamber when the processing unit determines that the predetermined dosage is available for withdrawal from the vaporizer.

20. The portable loose-leaf material vaporizer of claim 1, wherein the processing unit is configured to indicate to the user when a predetermined dosage is available for withdrawal from the vaporizer.

21. The portable loose-leaf material vaporizer of claim 1, wherein the mathematical model further relates temperature of the heating chamber to the dosage of the one or more active agents withdrawn or to be withdrawn from the vaporizer.

22. A portable loose-leaf material vaporizer, comprising:
a heating chamber for housing loose leaf material and vaporizing one or more active agents from the loose-leaf material,
a mouthpiece for withdrawal of the one or more active agents vaporized from the looseleaf material,
a processing unit, and
a flow detector for detecting flow through the vaporizer,
wherein the processing unit is configured to
control temperature of the heating chamber, and
estimate a dosage of the one or more active agents withdrawn from the vaporizer based on a mathematical model, and
wherein the mathematical model relates vapor generating time and looseleaf material properties to the dosage of the one or more active agents withdrawn or to be withdrawn from the vaporizer.

23. The portable loose-leaf material vaporizer of claim 22, wherein the flow detector is arranged outside a flow path connecting the heating chamber and the mouthpiece, wherein the flow detector is preferably arranged in a dead-end branch branching from the flow path connecting the chamber and the mouthpiece.

24. The portable loose-leaf material vaporizer of claim 22, wherein the flow detector is selected from the group consisting of differential pressure sensors, capacitive air flow sensors, spinning fans/turbines, moving flap-type sensors, temperature sensors and thermal flow sensors.

25. The portable loose-leaf material vaporizer of claim 22, wherein the mathematical model further relates temperature of the heating chamber to the dosage of the one or more active agents withdrawn or to be withdrawn from the vaporizer.

26. A portable loose-leaf material vaporizer, comprising:
a heating chamber for housing loose leaf material and vaporizing one or more active agents from the loose-leaf material,
a mouthpiece for withdrawal of the one or more active agents vaporized from the looseleaf material,
a processing unit, and
a flow regulating element configured to exert one among a plurality of resistances against flow through the vaporizer, and
optionally a flow detector for detecting flow through the vaporizer,
wherein the processing unit is configured to,
control temperature of the heating chamber, and
estimate a dosage of the one or more active agents withdrawn from the vaporizer based on a mathematical model, and
wherein the mathematical model preferably relates vapor generating time and looseleaf material properties and the one resistance exerted by the flow regulating element against the flow through the vaporizer, to the dosage of the one or more active agents withdrawn or to be withdrawn from the vaporizer.

27. Portable loose-leaf material vaporizer of claim 26, wherein the flow regulating element comprises a rotatable disk having at least a first section, which when rotated into a flow path of the vaporizer defines a first effective cross sectional flow area, and a second section, which when rotated into the flow path of the vaporizer defines a second effective cross sectional flow area different from the first cross sectional flow area.

28. The portable loose-leaf material vaporizer of claim 26, wherein the processing unit is further configured to control the resistance exerted by the flow regulating element against the flow through the vaporizer.

29. The portable loose-leaf material vaporizer of claim 26, further comprising:
a sensor for determining the resistance exerted by the flow regulating element against the flow through the vaporizer.

30. The portable loose-leaf material vaporizer of claim 26, further comprising:
one or more temperature sensors,
wherein the one or more temperature sensors are preferably located adjacent to the heating chamber and/or adjacent to the mouthpiece.

31. The portable loose-leaf material vaporizer of claim 26, wherein the vaporizer further comprises:
an interface for receiving sensor data and/or user input data,
wherein the interface for receiving user input data is optionally a user interface.

32. The portable loose-leaf material vaporizer of claim 26, wherein the mathematical model further relates temperature of the heating chamber to the dosage of the one or more active agents withdrawn or to be withdrawn from the vaporizer.

33. The portable loose-leaf material vaporizer of claim 26, wherein the sensor data and/or user input data include one or more of the following:
(i) a predetermined dosage of the one or more active agents to be withdrawn from the vaporizer; and/or
(ii) a number of draws to be taken from the vaporizer; and/or
(iii) the loose-leaf material properties; and/or
(iv) resistance exerted by a flow regulating element against the flow through the vaporizer; and/or
(v) a temperature of the heating chamber.
